(19) United States
(12) Patent Application Publication
(10) Pub. No.: US 2020/0246466 A1
(43) Pub. Date: Aug. 6, 2020

(54) ZWITTERIONIC GELS AND METHODS OF MAKING THE SAME

(71) Applicant: Colorado School of Mines, Golden, CO (US)

(72) Inventors: Melissa Krebs, Englewood, CO (US); Gulsu Sener, Portland, OR (US)

(73) Assignee: Colorado School of Mines, Golden, CO (US)

(21) Appl. No.: 16/781,197

(22) Filed: Feb. 4, 2020

Related U.S. Application Data
(60) Provisional application No. 62/800,959, filed on Feb. 4, 2019.

(51) Int. Cl. A61K 47/34; A61K 45/06; A61K 9/06; A61K 9/00

(52) U.S. Cl. CPC A61K 47/34; A61K 9/0024; A61K 9/06; A61K 45/06

(57) ABSTRACT

Zwitterionic cryogels and methods of making the same and using the same are disclosed. The zwitterionic cryogels are not brittle and are capable of self-healing by reestablishing crosslinks.

### CROSS REFERENCE TO RELATED APPLICATION

This application claims priority and the benefit under 35 U. S.C. § 119(e) to U.S. Provisional Patent Application Ser. No. 62/800,959 filed on Feb. 4, 2019, which is incorporated herein in its entirety by reference.

### FIELD OF THE INVENTION

The present invention relates to zwitterionic gels and methods of making the same.

### BACKGROUND

The skin is the largest organ of the human body, and protects the body from microbial invasion and other exterior damages. This organ, however, can be damaged (i.e. wounds), which are generated by mechanical or thermal damage. Wounds can be life threatening, depending on the size and depth of the wound. When wounds are difficult to heal, they can become chronic due to dysregulation of inflammation. Chronic wounds can be caused by, among other things, diabetes, burns, immunological states and vascular insufficiency. In diabetic patients, for example, healing impairment is caused by neuropathy, ischemia, and/or trauma. These factors can lead to opportunities for infections to populate the area, which may cause life-threatening infections. Hydrogels have long been considered as promising materials for wound dressing materials due to good oxygen permeation and a high water content that can help maintain a moist environment around the wound. More importantly, therapeutic molecules such as growth factors or antibiotics can be readily loaded into the hydrogels to promote wound healing and to protect the wound from bacterial infections and promote the healing process. Unfortunately, the weak, porous structure of hydrogels generally results in a rapid release of therapeutic molecules (typically a few hours) with a large burst release. Burst release of therapeutics not only decreases the efficiency of the therapy, but can also cause side effects due to the sudden increase in the blood concentrations of these molecules.

### SUMMARY

Zwitterionic hydrogels have demonstrated antifouling and antiadhesion characteristics, which often exceed PEGylated or PEG hydrogels, due to their closely repeating positive and negative charges. The most commonly explored zwitterionic monomers are [2-(methacryloloxy)ethyl]dimethyl-(3-sulfopropyl) ammonium hydroxide (SBMA), and 3-[[2-(Methacryloyloxy)ethyl]dimethylammonio]propionate (CBMA). These monomers have been polymerized using different zwitterionic or non-zwitterionic crosslinkers or co-polymerized with non-zwitterionic biocompatible monomers such 2-hydroxyethyl methacrylate (HEMA) and a crosslinker. Even in the presence of non-zwitterionic monomers or crosslinkers, these zwitterionic monomers still provide anti-fouling capabilities to hydrogels while fortifying the mechanical properties for a large range of applications. Controlled delivery of drugs has been investigated over extended timescales utilizing the charged pendant groups as a sequestration method for proteins. The polymer protects sequestered proteins from denaturation and maintains long-term bioactivity due to both the highly hydrated hydrophilic state and the zwitterions which provide a Hofmeister ion-like effect which stabilize protein conformations. In some instances, zwitterionic cryogels (i.e. hydrogels polymerized below freezing temperature conditions) can release proteins for several months with an almost constant rate. However, these chemically-crosslinked cryogels are tough and brittle which is not desirable for wound dressings.

An ideal wound dressing material should be able to sustainably release the loaded therapeutics for at least several weeks since large wounds may require long times to heal. In addition, such materials should be flexible and self-healable to resist crack formation due to mechanical stress generated during wound healing. In addition, good antifouling properties are required to decrease risk of infection at the wound by preventing bacterial adhesion and also to prevent cell adhesion which can cause sticking of the wound dressing to the wound. Furthermore, an injectable material would enable easy application to the area. Finally, wound dressing hydrogels should have low degradation rates to ensure protection of the wounded area during the entire healing process.

The present invention provides a suitable zwitterionic hydrogel material. The present invention relates to zwitterionic cryogels and methods of making the cryogels in the absence of any chemical crosslinker. The crosslinking of the polymerized gels is based around the hydrogen bonding and electrostatic attractions between pendant groups of the zwitterionic polymers. The resulting cryogels are very flexible and have demonstrated self-healing ability and injectability while maintaining their sustained drug release properties.

The hydrogel formulations of the present invention relate to three-dimensional networks of monomers that may be crosslinked by physical and/or chemical methods. The monomers may have two or more charged groups over a given pH range. In some embodiments, the polymers comprise zwitterionic monomers. As used herein, a zwitterionic monomer is any compound that is able to be polymerized and simultaneously includes both a positively and negatively charged group under physiological conditions. Characteristics (e.g., loading, elasticity, porosity, biodegradation rate, viscosity, antifouling properties, etc.) of these hydrogels can be modified by varying the concentration of monomer subunits.

Exemplary hydrogels are formed as copolymers comprising one or both of the zwitterionic monomers SBMA, methacryloyloxylethyl phosphorylcholine (MPC) and/or CBMA, and the non-zwitterionic monomer that include side chains containing polar or charged groups, which can include hydroxyethyl methacrylate (HEMA). In some embodiments, the hydrogel can be a polymerization of SMBA and HEMA, CBMA and HEMA, CBMA alone, or HEMA alone. One or more therapeutic agents can be added to the composition of monomers prior to polymerization. Notably, the monomers can be polymerized in the absence of any chemical crosslinker (e.g. glycerol dimethyacrylate (GDMA) , N, N methylbis (acrylamide) (MBA) , poly (ethylene glycol) diacrylates or methacrylates, ethylene glycoldimethacrylate (EGDMA) and combinations thereof). Rather, the polymerization of the monomers is initiated with the addition of ammonium persulfate (APS) and N,N,N′,N′-Tetramethylethylenediamine (TEMED) (in an amount between about 25 microliters and about 75 microliters to between about 12 mg/mL and about 20 mg/mL of APS solution and between about 0.5 microliters and 1 microliter of TEMED, in some embodiments 50 microliters of 13.6 mg/mL of APS solution and 0.85 microliter TEMED)

Polymerization can be performed in a temperature range between −80° C. and 50° C. When the polymerization is performed at a temperature below the freezing point of the aqueous phase of the gel solution, the resulting hydrogel can be referred to as a “cryogel” or “cryotopic gel.” Thus, these cryogels can be synthesized in semi-frozen liquid media in which ice crystals forming in the media act as porogen (pore generator) to create interconnected macro-pores after thawing. The shape and size of the ice crystals can contribute to the modification of the morphology and the porosity of the resulting cryogel. The polymerization temperature can be any temperature below the freezing point of the aqueous phase of the gel solution. Factors such as ratio of monomer subunits, polymerization temperature, rate of freezing, and solvent composition can be used to modify the characteristics of the hydrogel. For example, the internal pore size and density of the hydrogel can be varied by modifying the ratio of monomer subunits and the polymerization temperature. In most embodiments, the average pore size of the disclosed hydrogel can be between about 50 μm and about 100 μm.

In some embodiments, the polymerization can be performed at a temperature between about −30° C. and about 50° C., in some embodiments about −20° C. (thereby preparing cryogels). Other suitable polymerization temperatures include about −80° C., about −75° C., about −70° C., about −65° C., about −60° C., about −55° C., about −50° C., about −45° C., about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., or about 50° C., or any temperature range between two of these values. Polymerization can proceed for a time period between about 30 minutes and about 4 days. In some embodiments, the polymerization time can be about 30 minutes, about 1 hour, about 2 hours, about 5 hours, about 10 hours, about 15 hours, about 20 hours, about 24 hours, about 30 hours, about 35 hours, about 40 hours, about 45 hours, about 48 hours, about 55 hours, about 60 hours, about 65 hours, about 70 hours, about 75 hours, about 80 hours, about 85 hours, about 90 hours, about 95 hours, or about 96 hours, or any range between two of these values. One skilled in the art would understand that the duration of the polymerization will depend upon the temperature of the polymerization. In some embodiments, the polymerization can be conducted at a temperature of about −20° C. for a time period of about 24 hours. After polymerization, the cryogels can be thawed at room temperature (about 25° C.). The mole ratio of the zwitterionic monomer (SBMA, MPC and/or CBMA) to the non-zwitterionic monomer (HEMA) can range from 50:1 to 1:50. In some embodiments, the mole ratio of the zwitterionic monomer (SBMA, MPC and/or CBMA) to the non-zwitterionic monomer (HEMA) can be about 1:1. The polymerized hydrogels possess a macroporous structure with interconnected pores.

Following polymerization, these hydrogels can form a solid or semi-solid scaffold, a gel, a film, or a coating comprising a therapeutic agent(s). These hydrogels can be absorbent and can possess excellent antifouling properties and biocompatibility. The biocompatibility can be due to the hydrogel's high-water content and physiochemical similarity to native extracellular networks. These hydrogel formed from zwitterionic monomer (SBMA, MPC and/or CBMA) and a non-zwitterionic monomer (HEMA) are found to be mechanically stable after stretching or compression. They also demonstrate self-healing properties. Additionally, softer hydrogels comprising lower amounts of the zwitterionic monomers are softer and therefore may be suitable for administration by injection. Suitable therapeutic agents, loading of the therapeutic agents, and uses of the hydrogels with the therapeutic agents can be found in U.S. Publication No. 2018/0221490 (published Aug. 9, 2018), which is incorporated by reference in its entirety.

An aspect of the invention is a hydrogel. The hydrogel includes a polymerized zwitterionic monomers that includes one or more of a sulfobetaine methacrylate (SBMA) monomer, a methacryloyloxylethyl phosphorylcholine (MPC) or a carboxybetaine methacrylate (CBMA) monomer, polymerized with a hydroxyethyl methacrylate (HEMA) monomers.

An aspect of the invention is a method of making a hydrogel. The method includes mixing at least one zwitterionic monomer comprising at least one of a SBMA, a MPC or a CBMA, and a non-zwitterionic monomer HEMA monomers to form a mixture. Polymerization is initiated by the addition of a initiating polymerizing agent to the mixture. The polymerizing temperature is between about −20° C. and about 25° C. to form a hydrogel.

As aspect of the invention is a hydrogel incorporated into a bandage or dressing. The hydrogel includes at least polymerized zwitterionic monomer polymerized with at least one second monomer comprising HEMA.

### BRIEF DESCRIPTION OF THE FIGURES

FIG. 1A illustrates the FTIR spectra of the dried zwitterionic polymer of SMBA, HEMA, and the copolymer of SMBA and HEMA;

FIG. 1B illustrates the FTIR spectra of the dried zwitterionic polymer of CMBA, HEMA, and the copolymer of CMBA and HEMA.

FIG. 2A illustrates images of Sample 10;

FIG. 2B illustrates images of Sample 18;

FIG. 2C illustrates images of Sample 17;

FIG. 2D illustrates images of Sample 4;

FIG. 2E illustrates images of Sample 6;

FIG. 3 illustrates a S3c sample, which could be stretched to more than 5× 5its initial size without breaking;

FIG. 4A illustrates 100% HEMA sample under compression, illustrating that the sample could be highly compressed without any significant deformation;

FIG. 4B illustrates the compression of a cryogel prepared using only a HEMA monomer;

FIG. 5A illustrates Sample 9 with the two halves separated, then joined together;

FIG. 5B illustrates Sample 3 with two halves separated, then joined together;

FIG. 5C illustrates the lack of self-healing of a cryogel prepared using only a HEMA monomer;

FIG. 5D illustrates the storage modulus of a CH3c sample;

FIG. 5E illustrates the storage modulus of a CH6c sample;

FIG. 5F illustrates the storage modulus of a SH4c sample;

FIG. 6 illustrates a series of images taken as a sample is injected through the needle;

FIG. 7A illustrates the storage modulus (G′) for zwitterionic cryogels prepared at a 1:1 ratio of SBMA (three samples); FIG. 7B illustrates the loss modulus (G″) for zwitterionic cryogels prepared at a 1:1 ratio of SBMA (three samples);

FIG. 7C illustrates the storage modulus (G′) for zwitterionic cryogels prepared at a 1:1 ratio of CBMA (three samples);

FIG. 7D illustrates the loss modulus (G″) for zwitterionic cryogels prepared at a 1:1 ratio of CBMA (three samples);

FIG. 8A illustrates the storage modulus (G′) for cryogels prepared using different molar ratios of monomers (100 mole % HEMA; 25 mol % SBMA, 75 mol % HEMA; and 50 mol % SMBA, 50 mol % HEMA);

FIG. 8B illustrates the loss modulus (G″) for cryogels prepared using different molar ratios of monomers (100 mole % HEMA; 25 mol % SBMA, 75 mol % HEMA; and 50 mol % SMBA, 50 mol % HEMA);

FIG. 8C illustrates the storage modulus (G′) for cryogels prepared using different molar ratios of monomers (100 mole % HEMA; 25 mol % CBMA, 75 mol % HEMA; 50 mol % CBMA, 50 mol % HEMA; 75 mol % CBMA, 25 mol % HEMA; 100 mol % CBMA);

FIG. 8D illustrates the loss modulus (G″) for cryogels prepared using different molar ratios of monomer (100 mole % HEMA; 25 mol % CBMA, 75 mol % HEMA; 50 mol % CBMA, 50 mol % HEMA; 75 mol % CBMA, 25 mol % HEMA; 100 mol % CBMA);

FIG. 9A illustrates the storage modulus of the cryogel and room temperature gel over a variable frequency;

FIG. 9B illustrates the loss modulus of the cryogel and room temperature gels over a variable frequency;

FIG. 10 illustrates the GPC results;

FIG. 11A illustrates the storage modulus of CH1c samples after additional thaw freeze cycles;

FIG. 11B illustrates the loss modulus of CH1c samples after additional thaw freeze cycles;

FIG. 12 illustrates the mass of two samples—Sample 2 (SH2c) and Sample 9 (CH3c)—over time;

FIG. 13 illustrates degradation for Sample 2 and Sample 9; and

FIG. 14 illustrates the cell viability of several of the cryogels.

### DETAILED DESCRIPTION

The present invention relates to a hydrogel, and methods of making and using the same. An aspect of the invention is a hydrogel. The hydrogel includes zwitterionic monomers comprising one or more of SBMA monomer, MPC or CBMA monomer that are polymerized with HEMA monomers.

The ratio of the SBMA monomer to the HEMA monomer can be between about 50:50 and about 0:100. In some embodiments, the ratio of the CBMA monomer to the HEMA monomer can be between about 50:50 and about 0:100. In some embodiments, the ratio of MPC to HEMA can be between about 50:50 and about 0:100. In some embodiments, two of the monomers of SBMA, CBMA or MPC can be combined with HEMA. The ratio of the first monomer to the second monomer to the third monomer can be between about 0:1:1 to about 1:1:1. The mixture of the first monomer, second monomer, and/or third monomer can be combined with HEMA. The ratio of the mixture of the monomers to the HEMA can be between about 50:50 and about 0:100. The hydrogel can include a therapeutic agent. Suitable therapeutic agents can be found in U.S. Publication No. 2018/0221490, which is incorporated by reference in its entirety. The amount of therapeutic agent that can be included in the hydrogel is between about 0 wt. % and about 20 wt. %. In some embodiments, the amount of therapeutic agent can be about 0 wt. % about 5 wt. %, about 10 wt. %, about 15 wt. %, about 17 wt. % or about 20 wt. %, or any range between two of these values.

The hydrogel is a gel, and notably is not brittle. In some embodiments, the hydrogel can be a cryogel. The gel is not cytotoxic in some embodiments. Notably, in some embodiments, the hydrogel does not include a crosslinking agent. The hydrogel can be translucent, opaque, or transparent. In some embodiments, the hydrogel can swell to between about 3× and about 5× of its initial volume in between about 1 minute and about 3 days. In some embodiments, the hydrogel can stop swelling after a period of time between about 1 minute and about 15 days.

In some embodiments, the weight loss of the hydrogel is no more than about 30% after between about 5 days and about 42 days at a temperature between about 20° C. and about 40° C., in some embodiments about 37° C. In some embodiments, the weight loss can be between about 0% and about 30%, between about 10% and about 20%, or between about 5% and about 25%. In some embodiments, the weight loss can be about 1%, about 2%, about 5%, about 8%, about 10%, about 15%, about 20%, about 25%, or about 30%.

The storage modulus of the gel can be between about 1 Pa and about 10,000 Pa. In some embodiments, the storage modulus of the gel can be about 1 Pa, about 10 Pa, about 50 Pa, about 100 Pa, about 250 Pa, about 1000 Pa, about 2000 Pa, about 3000 Pa, about 4000 Pa, about 5000 Pa, about 6000 Pa, about 7000 Pa, about 8000 Pa, about 9000 Pa, or about 10000 Pa, or any range between two of these values. The loss modulus of the gel can be between about 1 Pa and about 1,000 Pa. In some embodiments, the loss modulus can be about 1 Pa, about 10 Pa, about 50 Pa, about 100 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, about 800 Pa, about 850 Pa, about 900 Pa, about 950 Pa, or about 1000 Pa, or any range between two of these values.

The gel can be stretched to between 0.5× and 20× of its original size without breaking at between about 20° C. and about 25° C. In some embodiments, the gels can establish crosslinking with another gel or reestablish a physical crosslink if the gel is cut, torn, damaged, etc. In some embodiments, full crosslinking can be reestablished (i.e. up to 100%). In some embodiments, the amount of reestablished crosslinking or newly established crosslinking can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or any value between two of these values. In comparison, when pure HEMA is used in the gel, 0% crosslinking is established or reestablished with a gel.

In some embodiments, the gel can be injectable. The gel can be injected through a 21 gauge or larger needle.

The hydrogels can include pores. In some embodiments, the porosity of the hydrogel can be between about 60% and about 96%. In some embodiments, the porosity can be about 60%, about 65%, about 79%, about 75%, about 80%, about 85%, about 90%, about 95%, or about 96%, or any value between two of the values. In some embodiments, pore can exist in the hydrogel of a size between about 50 μm and about 100 μm. The hydrogels can be used to produce a solid or semi-solid scaffold, a gel, a film, or a coating. An aspect of the invention is a method of making a hydrogel. The method includes mixing at least one zwitterionic monomer comprising at least one of SBMA, MPC or CBMA, and a non-zwitterionic monomer HEMA monomers to form a mixture. Polymerization of the monomers can be initiated by the addition of a polymerizing agent to the mixture at a temperature between about −80° C. and about 50° C. for between about 1 minute and about 4 days to form a hydrogel. The ratio of the zwitterionic monomer to the non-zwitterionic monomer can be between about 50:1 and about 1:50. In some embodiments, the polymerizing agent can be ammonium persulfate (APS), N,N,N,N-tetramethylethylenediamine (TEMED), riboflavin, riboflavin-5′-phosphate, and combinations thereof. In some embodiments, polymerization can be initiated with between about 0.85 μL and about 61 μL of a polymerizing agent per between about 0.05 mL and about 3.6 mL of the total reaction. In some embodiments, the polymerization agent can be between about 40 μL and about 60 μL of APS combined with between about 0.5 μL and about 1 μL of TEMED In some embodiments, about 50 μL of 13.6 mg/g of APS and 0.85 μL of TEMED can be used as the polymerizing agent per 0.45 mL total reaction. The concentration of the polymerizing agent can be between about 2 mg/mL and about 40 mg/mL, in some embodiments a concentration of about 13.6 mg/mL . In some embodiments, a crosslinking agent is not used.

In some embodiments, a therapeutic agent can be included in the mixture. When the therapeutic agent is included, the ratio of the therapeutic agent to the mixture can be between 0 wt. % of the therapeutic agent in the gel to about 20 wt. % of the therapeutic agent in the gel.

The temperature during polymerization can be between about −80° C. and about 50° C. In some embodiments the temperature during polymerization can be between about −20° and about 20° C. In some embodiments, the polymerization temperature can be about −20° C., about −15° C., about −10° C., about −5° C., about 0° C., about 5° C., about 10° C., about 15° C., or about 20° C. In some embodiments when the polymerization occurs at a temperature less than about 20° C., the gel can be thawed to a temperature of between about 20° C. and about 30° C. The rate of freezing can be between about 0.5° C./min and about 5° C./min, in some embodiments about 1° C./min.

In some embodiments, the zwitterionic monomer can be dissolved in a liquid. The liquid can be water (distilled, deionized, tap, or combinations thereof), or combinations thereof. Table 1 below provides values of suitable concentrations of the monomer, and one skilled in the art would understand that a range between these values would also be acceptable. The reaction mixture can be put into a mold to produce a hydrogel product having certain characteristics.

The gel can be incorporated or impregnated into a bandage or dressing. In some embodiments, the bandage or dressing can be applied to a wound.

### EXAMPLES

### Gel Preparation of CNP-146a Hydrogel Compositions From Zwitterionic Monomers

Copolymer hydrogels were prepared from different combinations of two different zwitterionic monomers. SBMA was purchased from AFG Bioscience, CBMA was purchased from TCI America, HEMA, APS, and TEMED were purchased from Acros Organics. Phosphate buffered saline (PBS) with calcium and magnesium was purchased from HyClone. All chemicals were used as received.

### Gel Preparation

Zwitterionic gels were prepared by dissolving appropriate amounts of SBMA or CBMA and 2-hydroxyethyl methacrylate (HEMA) in 0.45 mL water. The monomer concentrations used are given in Tables 1 and 2. Polymerization was initiated using 50 μL of 13.6 mg/mL APS solution and 0.85 μL TEMED, and the reaction mixtures were poured into plastic molds (3 mL syringe with inner diameter 0.5 cm or 12 well tissue culture plate) and polymerized at room temperature or −20° C. for 24 hr. Cryogels were thawed at room temperature (about 25° C.) after polymerization was complete.

### Characterization

### Rheology Measurements

Gel samples of about 22 mm diameters and about 4 mm in height were prepared in 12 well plates. To determine the rheological properties of the gels, frequency sweep and strain sweeps test were performed using an AR-G2 rheometer (TA Instruments) equipped with a 20mm diameter crosshatched parallel plate at 37° C. Frequency sweep measurements were performed at 1% strain.

Strain sweep measurements were performed at 10 rad/s frequency. For FTIR measurements gel samples were lyophilized and dried powders were analyzed using a Nexus 470 ESP FT-IR Spectrometer equipped with an ATR accessory (Specac, Golden Gate).

### Swelling Tests

To determine the swelling behavior of the zwitterionic hydrogel, as-prepared gels were weighed to determine the mass of initial samples (mᵢₙᵢₜᵢₐₗ), soaked in PBS (pH:7.4), and allowed to swell in an incubator at 37° C. The hydrogels were taken at selected time intervals and transferred from one petri dish to another several times to remove excess water from the hydrogel surface, and then weighed (m_{wet gel}) to determine the swelling of the gels.

### Degradation Tests

To determine the degradation behavior of the hydrogels, gels were soaked in PBS (pH 7.4) and allowed to sit in an incubator at 37° C. The hydrogels were taken at selected time intervals, lyophilized, and weighed to determine the degradation percentage at different time intervals.

### Cytotoxicity

Cells were co-cultured with the zwitterionic gels to determine whether the materials exhibited any cytotoxicity. Briefly, 0.5 mL gels were fabricated and each was rinsed in 5 mL PBS for 24 h. 100 μL of each of these gels was placed in a Transwell membrane for a 24 well plate. Each well was sterilized briefly with 70% ethanol for 2 h and then rinsed with PBS three times. After sterilization MC3T3 cells were seeded into each well and cultured for 24 h. Cell viability was measured using a cell counting kit 8 (Dojindo) and normalized by comparing with cells cultured on tissue culture plastic.

### X-Ray Photoelectron Spectroscopy Analysis

X-ray photoelectron spectroscopy (XPS) measurements were performed on an ESCALAB-250Xi spectrometer in an ultra-high vacuum (UHV) chamber (below 7×10⁻⁹ mbar) using a monochromatic Al—K αradiation source. The spot size of the beam was 250 μm. C 1 s peak at 284.6 eV was used as a reference for calibration and Avantage Peakfit® software was used for peak fitting to identify the chemical functional groups present in gel derivatives.

### Gel Preparation of Copolymer Hydrogels Using Different Zwitterionic Monomers.

### Materials

To prepare the copolymer hydrogels, different combinations of two zwitterionic monomers; sulfobetaine methacrylate (SBMA) or carboxybetaine methacrylate (CBMA), and a non-zwitterionic monomer; hydroxyethyl methacrylate (HEMA) were polymerized in the absence of any chemical crosslinker.

### Gel Preparation

To initiate polymerization ammonium persulfate (APS) and N,N,N′,N′-Tetramethylethylenediamine (TEMED) couple were used and polymerizations were performed either at room temperature or −20° C. (to prepare cryogels) for 24 h. After polymerization, cryogels were thawed at room temperature (about 25° C.).

In initial experiments, the mole ratio of zwitterionic monomer (SBMA or CBMA) to the non-zwitterionic HEMA monomer were kept constant (1:1), but total monomer amounts were varied. All compositions and polymerization conditions are summarized in Table 1.

In general, five different types of materials were obtained after polymerization: a transparent gel, a translucent gel, an opaque gel, a viscous liquid, and a solution (i.e., no gelation). Table 1 sets forth compositions and polymerization conditions of copolymers prepared 1:1 mole ratio of zwitterionic (CBMA or SBMA) and non-zwitterionic monomer (HEMA). Polymers prepared using pure monomers for control experiments are also given at the bottom of the table. The sample nomenclature is as follows: the uppercase letters indicate the zwitterionic monomer (‘S’ or ‘C’ for SBMA or CBMA, respectively) and the non-zwitterionic monomer (‘H’ for HEMA), respectively, that was used in the synthesis. The number indicates the sample number prepared using the same monomers and polymerization temperature. Finally, the lowercase letter(s) indicates the polymerization temperature (‘rt’ for room temperature and ‘c’ for −20° C.). The values in Table 1 are approximate.

TABLE 1

| Sample | | SBMA | CBMA | HEMA | Temp. | |
|---|---|---|---|---|---|---|
| # | Sample | (mg/mL) | (mg/mL) | (mg/mL) | (° C.) | Appearance |
| 1 | SH1c | 60 | — | 27 | −20 | Gel (translucent) |
| 2 | SH2c | 90 | — | 40.5 | −20 | Gel (translucent) |
| 3 | SH3c | 150 | — | 67.5 | −20 | Gel (transparent) |
| 4 | SH1rt | 60 | — | 27 | 20 | Solution |
| 5 | SH2rt | 90 | — | 40.5 | 20 | Solution |
| 6 | SH3rt | 150 | — | 67.5 | 20 | Viscous liquid |
| 7 | CH1c | — | 24 | 13.5 | −20 | Gel (translucent) |
| 8 | CH2c | — | 48 | 27 | −20 | Gel (translucent) |
| 9 | CH3c | — | 72 | 40.5 | −20 | Gel (translucent) |
| 10 | CH4c | — | 144 | 81 | −20 | Gel (transparent) |
| 11 | CH1rt | — | 24 | 13.5 | 20 | Solution |
| 12 | CH2rt | — | 48 | 27 | 20 | Solution |
| 13 | CH3rt | — | 72 | 40.5 | 20 | Solution |
| 14 | CH4rt | — | 144 | 81 | 20 | Gel (transparent) |
| 15 | S1c | 150 | — | — | −20 | Solution |
| 16 | C1c | — | 144 | — | −20 | Gel (translucent) |
| 17 | H1c | — | — | 81 | −20 | Gel (opaque) |

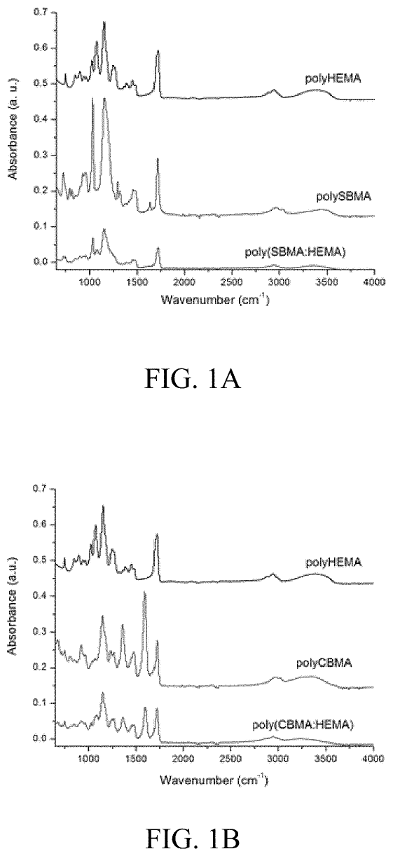

Because no chemical crosslinker was used, the gel formation can be attributed to the strong physical interactions between the zwitterionic side groups of the polymers. In addition, hydroxyl side groups of the polymer can form hydrogen bonds which can further improve the interactions between two polymer chains. Interestingly, all the formulations (Table 1) that were polymerized at cryoconditions (−20° C.) formed gels, but when the same compositions were polymerized at room temperature gel formation was only observed for Sample 14, where high amounts of CBMA (144 mg/mL) was used. This formation was found over a range of compositions of the zwitterionic monomer (between about 24 mg/mL and about 150 mg/mL) combined over a range of the non-zwitterionic monomer (between about 13.5 mg/mL and about 81 mg/mL). For the SBMA monomer, room temperature polymerization did not yield gelation even for Sample 6 with a high monomer concentration of 150 mg/mL. For control experiments, solutions that contained only SBMA (Sample 15), CBMA (Sample 16), and HEMA (Sample 17) were polymerized at −20° C. Gel formation was not observed for the SBMA polymerization solutions even at a monomer concentration of 150 mg/mL. Alternatively, CBMA formed gels at a concentrations of 144 mg/mL, but the gels were much weaker compared to the gels prepared using both CBMA and HEMA monomers. Polymerization of HEMA solutions (81 mg/mL) also resulted in a formation of an opaque gel. The chemical composition of the polymers and copolymers were characterized using FTIR spectroscopy (FIG. 1). FIG. 1A illustrates the FTIR spectra of the dried zwitterionic polymer of SMBA, HEMA, and the copolymer of SMBA and HEMA. FIG. 1B illustrates the FTIR spectra of the dried zwitterionic polymer of CMBA, HEMA, and the copolymer of CMBA and HEMA.

The FTIR adsorption peaks corresponding to both monomers were present in the FTIR spectra demonstrating that both monomers were present in the copolymers.

### Visual Inspection

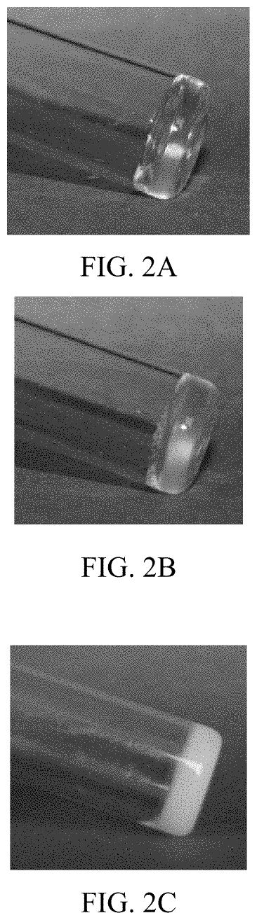

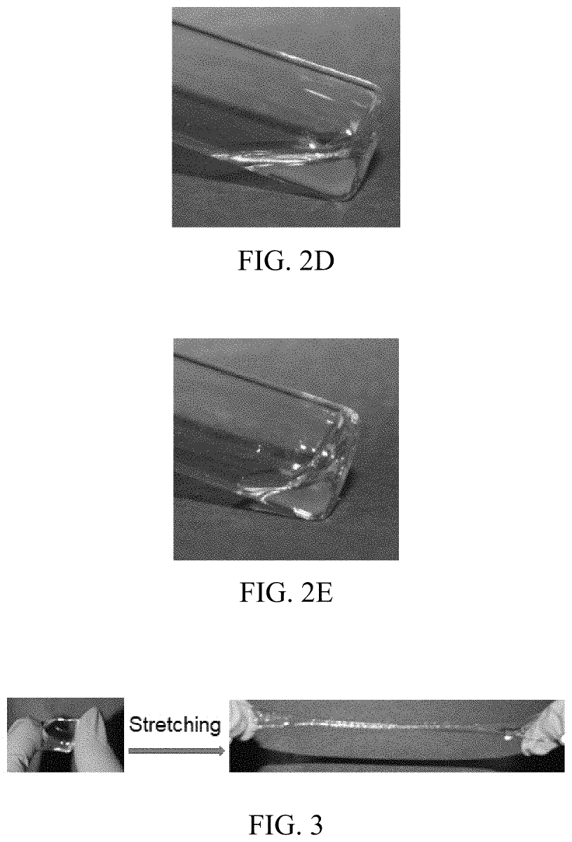

FIGS. 2A-E illustrate polymerization of several samples. FIG. 2A illustrates images of Sample 10. FIG. 2B illustrates images of Sample 18. FIG. 2C illustrates images of Sample 17. FIG. 2D illustrates images of Sample 4. FIG. 2E illustrates images of Sample 6.

### Mechanical Stability

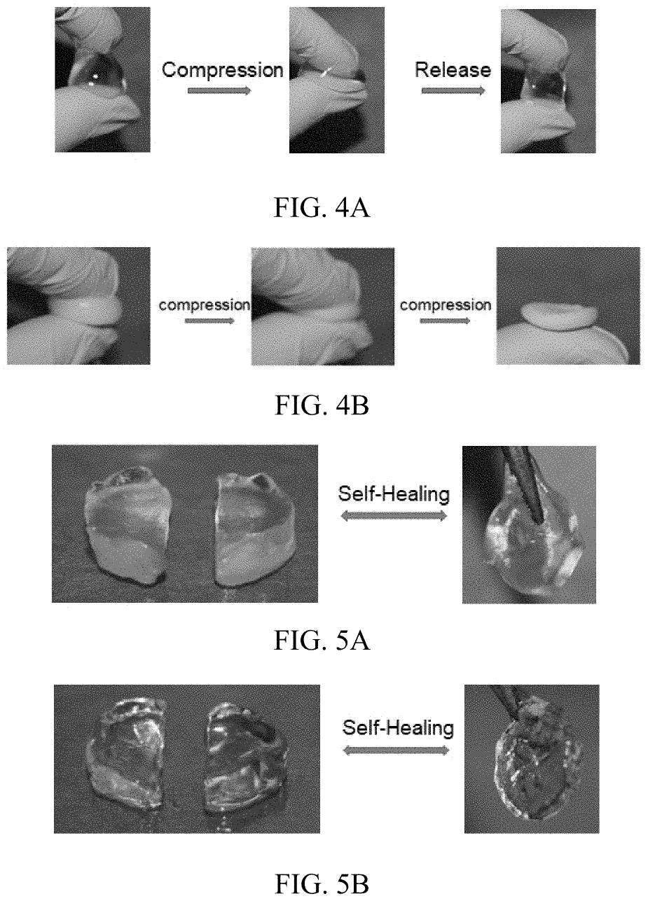

The gels containing zwitterionic monomers were found to be mechanically stable after stretching or compression. FIG. 3 illustrates a S3c sample, which could be stretched to more than 5× its initial size without breaking. FIG. 4A illustrates 100% HEMA sample under compression, illustrating that the sample could be highly compressed without any significant deformation. FIG. 4B illustrates the compression of a cryogel prepared using only a HEMA monomer. Once a force is applied, the HEMA cryogel compresses and remains compressed.

### Reestablish Cross-Link

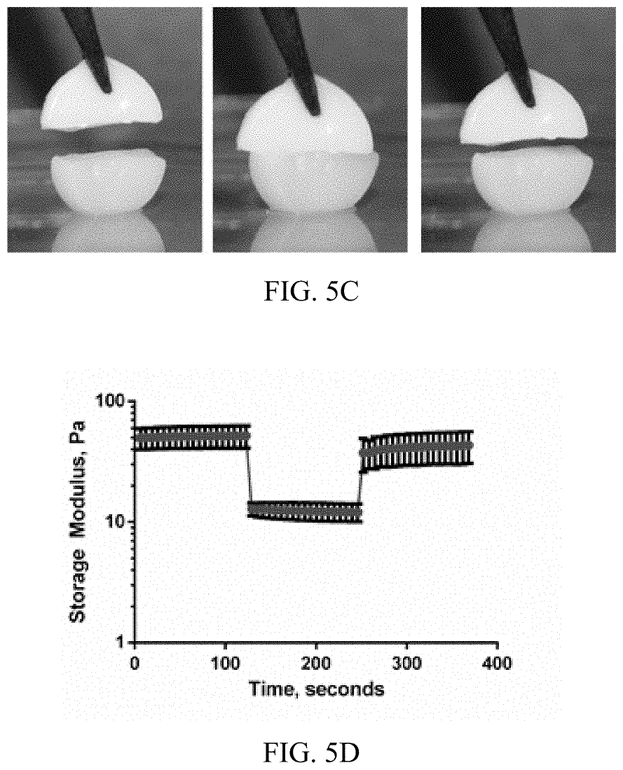

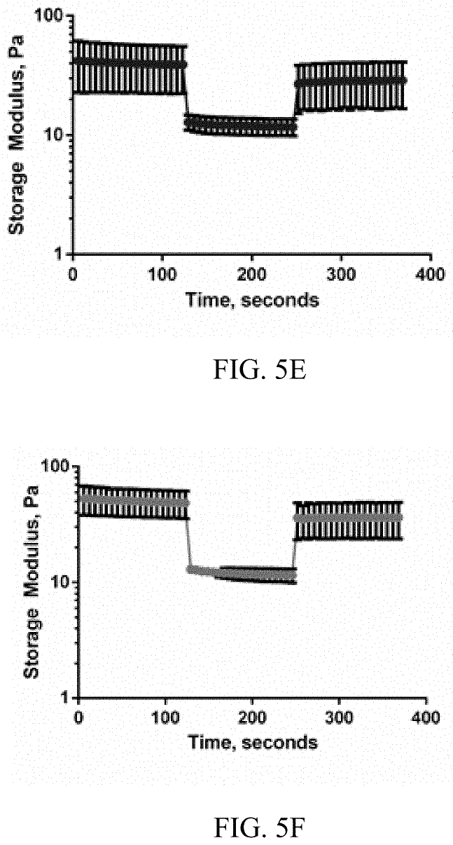

To determine whether the physical crosslinks of the gels could be reestablished after breakage, two Sample 9 or Sample 3 cryogels were prepared, then cut into two pieces. FIG. 5A illustrates Sample 9 with the two halves separated, then joined together. FIG. 5B illustrates Sample 3 with two halves separated, then joined together. One of the two gel pieces was colored red using Alizarin Red S dye (25 μg/mL). One colored and one uncolored piece of each gel were then brought together and stretched to determine if they self-healed. Self-healing of both gels was observed almost immediately. Note that even when a gravitational force was exerted on the reconnected samples, that the samples remained connected.

By comparison, cryogels prepared using only HEMA monomers were highly deformed after compression and could not recover initial shapes after compression. FIG. 5C illustrates the lack of self-healing of a cryogel prepared using only a HEMA monomer. Additionally, self-healing was not observed for the pure HEMA cryogels and they were not injectable. These results suggest that the zwitterionic side groups of the polymer provides good mechanical properties and self-healing abilities to the cryogels.

FIGS. 5D-5F illustrate self-healing capability of three of the zwitterionic cryogel formulations. The storage modulus at low shear was first measured, then the gels were exposed to high shear where the gel broke down and the storage modulus decreased, and when the shear was again reduced the gels were able to self-heal and exhibit their pre-high-shear strength. FIG. 5D illustrates the storage modulus of a CH3c sample. FIG. 5E illustrates the storage modulus of a CH6c sample. FIG. 5F illustrates the storage modulus of a SH4c sample.

### Cryogel Injectability

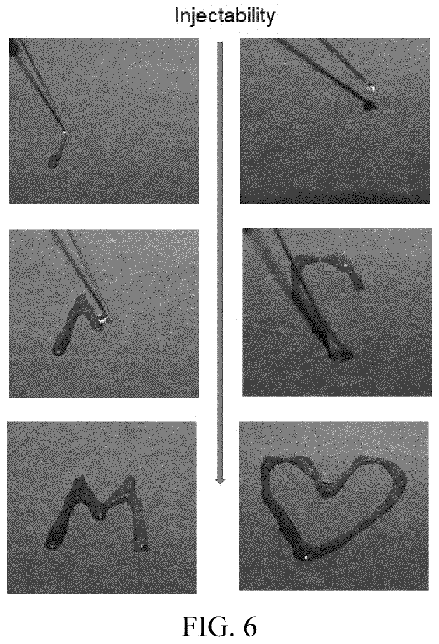

Softer cryogels, prepared using lower monomer amounts (for example, the Sample 1 gel), could be injected from a 21 gauge needle. FIG. 6 illustrates a series of images taken as a sample is injected through the needle.

### Viscoelastic Properties

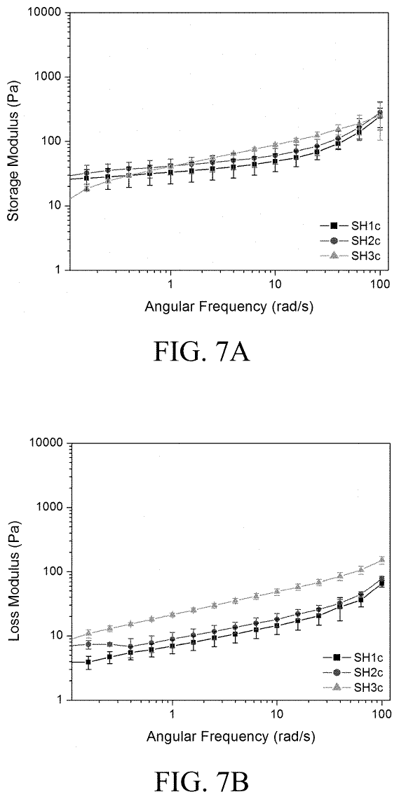

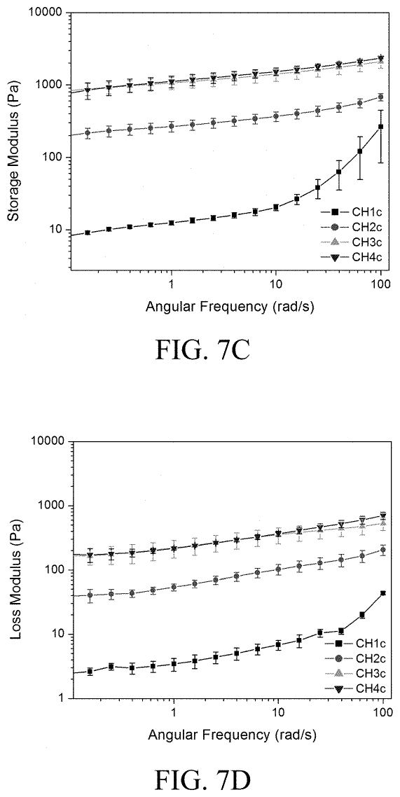

Viscoelastic properties of the zwitterionic cryogels were investigated using rheometry to better evaluate their mechanical properties. FIG. 7 illustrates rheology of the zwitterionic cryogels that were prepared using 1:1 mole ratio of SBMA or CBMA, and HEMA monomers. FIG. 7A illustrates the storage modulus (G′) for zwitterionic cryogels prepared at a 1:1 ratio of SBMA (three samples). FIG. 7B illustrates the loss modulus (G″) for zwitterionic cryogels prepared at a 1:1 ratio of SBMA (three samples). FIG. 7C illustrates the storage modulus (G′) for zwitterionic cryogels prepared at a 1:1 ratio of CBMA (three samples). FIG. 7D illustrates the loss modulus (G″) for zwitterionic cryogels prepared at a 1:1 ratio of CBMA (three samples).

Larger storage modulus (G′) values than loss modulus (G″) values were observed for all of the zwitterionic cryogels indicating their viscoelastic property. In addition, higher G′ and G″ were observed for the CBMA hydrogels indicating better mechanical properties of these gels. Stronger gel formation is expected for the gels prepared using the SBMA monomer because it has a greater difference between ionic strengths of the zwitterionic subunits, which should yield stronger ionic interactions between the polymer chains. However, the opposite result was observed for these cryogels. It should be noted that other than with increasing ionic strength difference, the physical interactions between polymer chains should become stronger with the increasing average molecular weight of the polymer, which could be the reason for the stronger gel formation with CBMA monomers compared to SBMA monomers.

Higher G′ and G″ values were also observed for the CBMA gels as the total monomer amount was increased up to a CBMA concentration of 72 mg/mL (FIGS. 7C and 7D). Further, increasing the CBMA concentration to 144 mg/mL did not significantly improve the mechanical properties of the zwitterionic cryogels. Alternatively, increasing the SBMA monomer concentration did not significantly affect the mechanical properties of the gels (FIGS. 7A and 7B); there was only a slight improvement in G′ and G″ values even after using a high SBMA concentration of 150 mg/mL.

### Gel Preparation—Vary Mole Ratio of Monomers

To understand the effect of monomer mole ratios on the mechanical properties of the hydrogels, the mole ratio of the monomers was varied while keeping the total monomer amount constant. The polymerized compositions were summarized in Table 2. The values in Table 2 are approximate.

TABLE 2

| Sample | | SBMA | CBMA | HEMA | Temperature | |
|---|---|---|---|---|---|---|
| # | Sample | (mg/mL) | (mg/mL) | (mg/mL) | (° C.) | Appearance |
| 18 | CH5c (75% CBMA) | — | 108 | 20.3 | −20 | Gel (translucent) |
| 19 | CH6c (25% CBMA) | — | 36 | 60.8 | −20 | Gel (opaque) |
| 20 | CH5rt (75% CBMA) | — | 108 | 20.3 | 20 | Solution |
| 21 | CH6rt (25% CBMA) | — | 36 | 60.8 | 20 | Solution |
| 22 | SH4c (25% SBMA) | 45 | — | 61 | −20 | Gel (opaque) |
| 23 | SH5c (75% SBMA) | 135 | — | 20.5 | −20 | Solution |

As noted above, the polymerization compositions that contain 100 mole % SBMA monomer did not form gels in any of the tested conditions, but it was possible to prepare hydrogel using pure HEMA and CBMA monomers. Additionally, the solution that contains 75 mole % SBMA did not form a gel, but all other copolymer compositions formed gels when polymerized at cryoconditions.

### Modulus

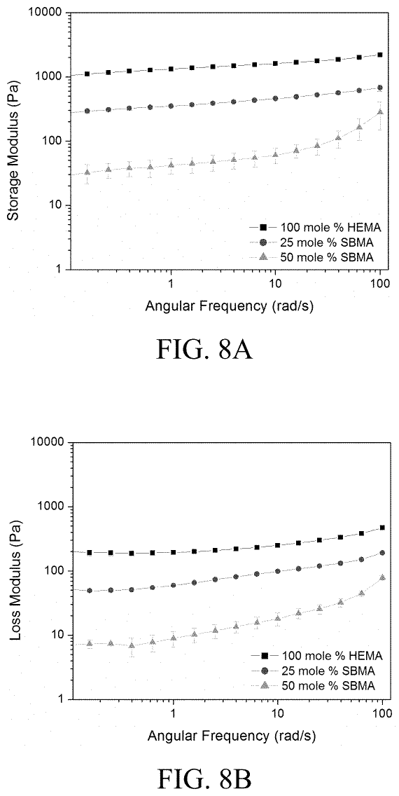

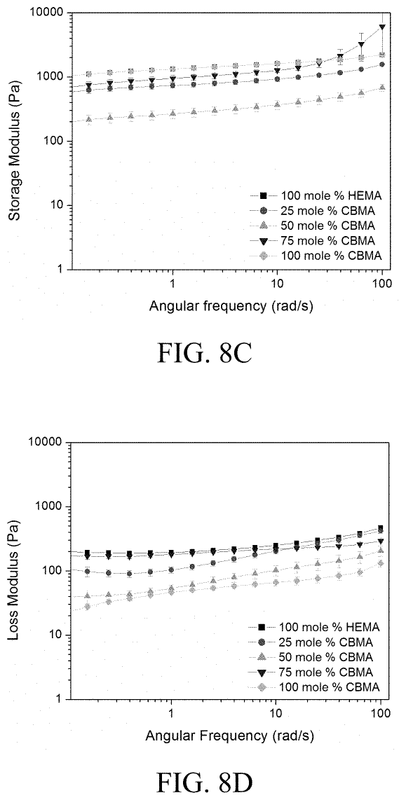

The compositions that yielded zwitterionic hydrogel were then characterized through rheology measurements (illustrated in FIGS. 8A-8D). FIG. 8A illustrates the storage modulus (G′) for cryogels prepared using different molar ratios of monomers (100 mole % HEMA; 25 mol % SBMA, 75 mol % HEMA; and 50 mol % SMBA, 50 mol % HEMA). FIG. 8B illustrates the loss modulus (G″) for cryogels prepared using different molar ratios of monomers (100 mole % HEMA; 25 mol % SBMA, 75 mol % HEMA; and 50 mol % SMBA, 50 mol % HEMA). FIG. 8C illustrates the storage modulus (G′) for cryogels prepared using different molar ratios of monomers (100 mole % HEMA; 25 mol % CBMA, 75 mol % HEMA; 50 mol % CBMA, 50 mol % HEMA; 75 mol % CBMA, 25 mol % HEMA; 100 mol % CBMA). FIG. 8D illustrates the loss modulus (G″) for cryogels prepared using different molar ratios of monomer (100 mole % HEMA; 25 mol % CBMA, 75 mol % HEMA; 50 mol % CBMA, 50 mol % HEMA; 75 mol % CBMA, 25 mol % HEMA; 100 mol % CBMA). Pure HEMA and CBMA hydrogel showed the highest and lowest G′ and G″, respectively, and copolymer hydrogel demonstrated mechanical properties between those of pure hydrogel. In general, it was observed that increasing the HEMA monomer mole percentage resulted in higher G′ and G″ values. The only exception was the hydrogel prepared using 75 mole % CBMA (Sample 23), which yielded higher G′ and G″ values than 50 mole % CBMA containing hydrogel.

### Rheology

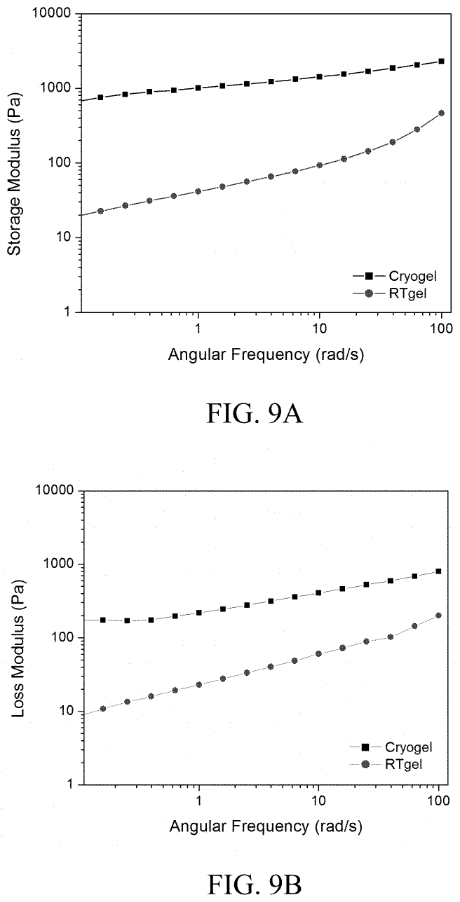

Rheology characterization was also performed for the composition that yielded hydrogel formation using room temperature polymerization, Sample 14 (CH4rt), and compared it with its cryogel counterpart (illustrated in FIGS. 9A and 9B). FIG. 9A illustrates the storage modulus of the cryogel and room temperature gel over a variable frequency. FIG. 9B illustrates the loss modulus of the cryogel and room temperature gels over a variable frequency. While the gels formed at room temperature were very weak, around 10-fold higher G′ and G″ values were measured for the same composition polymerized using cryoconditions, indicating that polymerization under cryoconditions improves the mechanical properties of the gels. There are several possible reasons for the improved mechanical properties for the gels polymerized at cryoconditions compared to those prepared at room temperature. First, the cryoconcentration effect, which is locally enhanced monomer concentrations due to the phase separation of the monomer phase and water phase during freezing, may result in the formation of a denser polymer network than their counterparts, which were prepared at room temperature. Second, in addition to a denser network at cryoconditions, longer polymer chains (higher average molecular weight) may be formed due to the reduced polymerization rate, which can improve the interactions between polymer chains and, thus, improve the mechanical properties of the gels. Finally, the freeze-thaw process may improve the mechanical properties of the gels as observed previously for PVA-based physically-crosslinked hydrogels.

### GPC Analysis

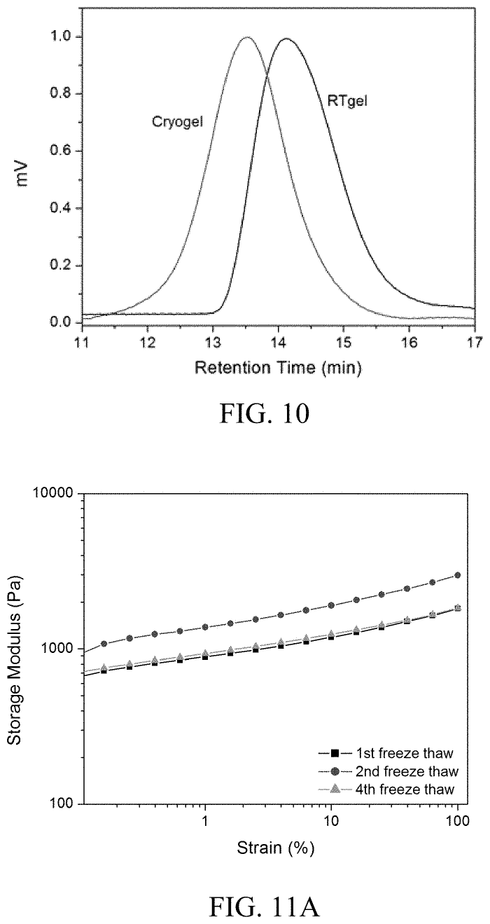

While there is no method to directly observe the cryoconcentration effect, the contribution of the other possible reasons can be tested. To see if the formed chains have higher molecular weights when the same composition was polymerized at lower temperatures, gel permeation chromatography (GPC) analysis was performed on two poly(SBMA:HEMA) polymers prepared using the same polymerization solution but at different temperatures (room temperature, and −20 C). To prevent gelation, the monomer amounts were kept low (0.04 mg/mL of SBMA and 0.015 mg/mL of HEMA). FIG. 10 illustrates the GPC results. FIG. 10 and Table 3 illustrate a significantly higher molecular weight for the polymer prepared at cryoconditions.

TABLE 3

| | Retention time | Area | Area | | | | |
|---|---|---|---|---|---|---|---|
| | [min] | [mV*sec] | [%] | Mn | Mw | Mz | Mw/Mn |
| CRYOGEL | 13.528 | 2927.025 | 100 | 55500 | 100371 | 176746 | 1.808 |
| RT GEL | 14.122 | 2982.973 | 100 | 22110 | 34934 | 48175 | 1.58 |

### Cryo Characteristics

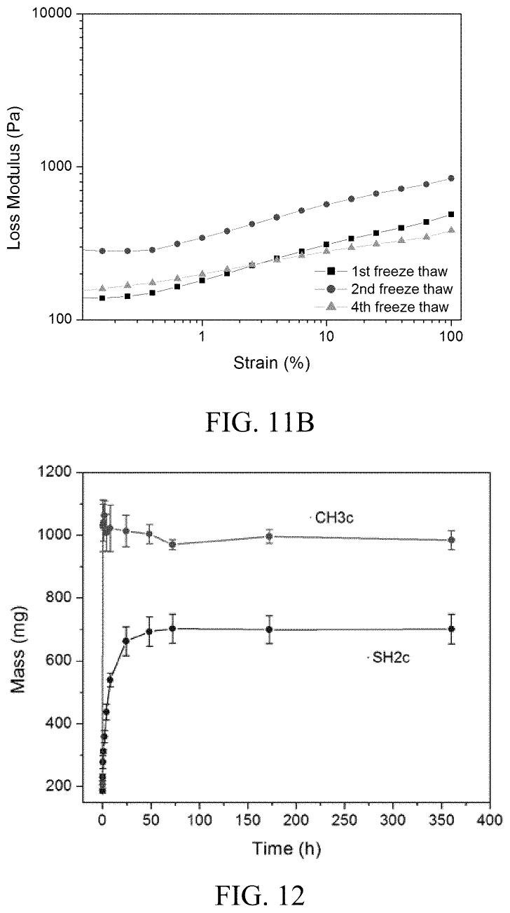

To determine whether the freezing and thawing of the gel contributes to the improved mechanical properties of the cryogels, additional freeze-thaw cycles were performed on the Sample 7 (CH1c) samples, and rheology measurements performed afterwards. FIG. 11A illustrates the storage modulus of CH1c samples after additional thaw freeze cycles. FIG. 11B illustrates the loss modulus of CH1c samples after additional thaw freeze cycles. After 4 freeze-thaw cycles, there was no significant change in the storage or loss moduli of the gels. These results suggest that the main reason for improved mechanical properties of the cryogels is due to the polymer formation with higher average molecular weight during polymerization, which should yield stronger interactions between the polymer chains. In addition, denser polymer network formation due to the cryoconcentration effect may improve the mechanical properties of the gels.

### Swelling Characteristics

The swelling property of the copolymer cryogels in PBS was also studied. FIG. 12 illustrates the mass of two samples—Sample 2 (SH2c) and Sample 9 (CH3c)—over time. Sample 2 gradually swelled to around 3.5× its initial volume in 2 days and the volume did not change after further incubation in PBS for 13 days. Alternatively, for Sample 9, swelling was very rapid. This gel swelled to around 5× of its initial volume in a few minutes and its volume did not change after that for a period of 15 days.

### Gel Degradation

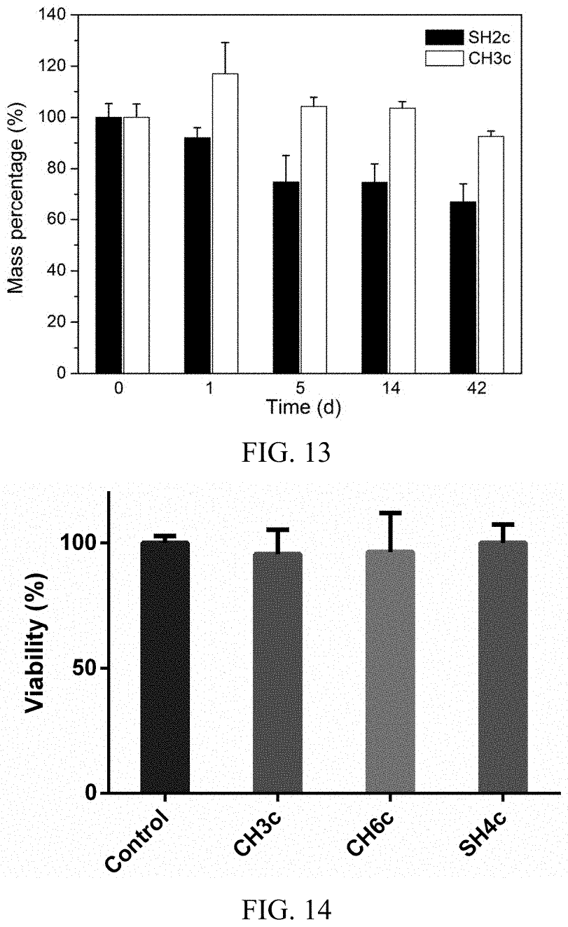

To study the possible degradation of these physically crosslinked cryogels, the SH2c and CH3c samples were incubated in PBS at 37° C., and at different time intervals, some of the cryogels were dried and weight of the dry products was measured. It was observed that after 42 days, the weight loss in Sample 2 was more than 30%, but only 8% for Sample 9. FIG. 13 illustrates degradation for Sample 2 and Sample 9. The lower degradation rate of the CBMA-based cryogels may be attributed to their stronger interactions between the polymer chains.

### Cytotoxicity

To ensure that the cryogels are not cytotoxic after being formed (i.e. due to the presence of unreacted monomers), MC3T3 cells were cultured in the presence of three different zwitterionic cryo-gel compositions. It was found that for all three types of gels, there was no impact on cell viability. FIG. 14 illustrates the cell viability of several of the cryogels.

Ranges have been discussed and used within the forgoing description. One skilled in the art would understand that any sub-range within the stated range would be suitable, as would any number within the broad range, without deviating from the invention. Further, when a list of specific numbers is given for a particular property, one skilled in the art would understand that each specific number can be used to provide a range without deviating from the invention.

The foregoing description of the present invention, related to zwitterionic gels and methods of making the same, has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A hydrogel, comprising:
polymerized zwitterionic monomers comprising one or more of a sulfobetaine methacrylate (SBMA) monomer, a methacryloyloxylethyl phosphorylcholine (MPC) or a carboxybetaine methacrylate (CBMA) monomer; and
polymerized a hydroxyethyl methacrylate (HEMA) monomers.

2. The hydrogel of claim 1, wherein the hydrogel further comprises a therapeutic agent.

3. The hydrogel of claim 1, wherein the hydrogel is a cryogel.

4. The hydrogel of claim 1, wherein the hydrogel does not include a crosslinking agent.

5. The hydrogel of claim 1, wherein the hydrogel is translucent, transparent or opaque.

6. The hydrogel of claim 1, wherein the hydrogel swells to between 300%and 500% of an initial volume of the hydrogel in between 1 minute and 3 days at a temperature of about 37° C.

7. The hydrogel of claim 1, wherein the hydrogel consists of SBMA and HEMA.

8. The hydrogel of claim 1, wherein the hydrogel stretches to between 0.5× and 20× of an original dimension without breaking.

9. The hydrogel of claim 1, wherein the hydrogel is capable of establishing a physical crosslink with a second material or a damaged part of the hydrogel.

10. The hydrogel of claim 1, wherein the hydrogel is injectable though a 21 gauge or larger needle.

11. A method of making a hydrogel, comprising:
mixing at least one zwitterionic monomer comprising at least one of a sulfobetaine methacrylate (SBMA), a methacryloyloxylethyl phosphorylcholine (MPC) or a carboxybetaine methacrylate (CBMA), and a non-zwitterionic monomer hydroxyethyl methacrylate (HEMA) monomers to form a mixture;
initiating the polymerization of the monomers in the composition by the addition of a initiating polymerizing agent to the mixture;
polymerizing the monomers in the mixture at a temperature between about −20° C. and about 25° C. to form a hydrogel.

12. The method of claim 11, wherein the temperature is about −20° C.

13. The method of claim 5, further comprising at least one therapeutic agent in the mixture.

14. The method of claim 11, wherein a ratio of the at least one zwitterionic monomer to the non-zwitterionic monomer is between about 50:1 and about 1:50.

15. The method of claim 14, wherein the polymerization does not include a crosslinking agent.

16. The method of claim 11, wherein the initiating polymerizing agent is an APS, a TEMED, and combinations thereof.

17. The method of claim 11, wherein a concentration of the initiating polymerizing agent is between about 13 mg/mL and about 150 mg/mL.

18. The method of claim 11, wherein a ratio of the mixture to the initiating polymerizing agent is between 0.5 μL and about 70 μL per 0.45 ml of the mixture.

19. The method of claim 11, further comprising thawing the hydrogel to a temperature between about 20° C. and about 30° C.
